(19) United States
(12) Patent Application Publication
(10) Pub. No.: US 2024/0180510 A1
(43) Pub. Date: Jun. 6, 2024

(54) SYSTEM AND METHOD FOR MEASURING VESSELS IN A BODY

(71) Applicants: William E. Butler, Cambridge, MA (US); AngioWave Imaging, Inc., Concord, MA (US)

(72) Inventors: William E. Butler, Cambridge, MA (US); Hector Andrade-Loarca, Munich (DE)

(21) Appl. No.: 18/526,233

(22) Filed: Dec. 1, 2023

Related U.S. Application Data
(60) Provisional application No. 63/429,606, filed on Dec. 2, 2022.

(51) Int. Cl. A61B 6/50; G06T 7/00; G06T 7/11; G06T 7/62

(52) U.S. Cl. CPC A61B 6/504; G06T 7/0012; G06T 7/11; G06T 7/62; G06T 2207/20081; G06T 2207/30101; G06T 2211/404

(57) ABSTRACT

Methods and systems are provided for measuring a vessel in a body. In one example, a computer obtains at least one angiographic image frame. The computer identifies pixels corresponding to a vessel of a body in the at least one angiographic image frame. The computer further identifies a medial axis of the vessel, and determines a measurement of the vessel in the at least one angiographic image frame by counting pixels in a transverse direction relative to the medial axis of the vessel and converting the dimension in pixels to a dimension in units of size.

### CROSS-REFERENCE TO RELATED APPLICATION

This application claims priority to U.S. Provisional Patent Application No. 63/429,606, filed on Dec. 2, 2022, the disclosure of which is incorporated by reference herein in its entirety.

### FIELD

The present disclosure relates generally to angiography, and more specifically to a system and method for measuring the dimensions of vessels in angiographic images.

### BACKGROUND

In coronary angiography, a catheter is advanced to the coronary arteries, radiopaque contrast is injected via the catheter, and a sequence of x-ray images is fluoroscopically obtained. The pattern filled by the radiopaque contrast on the resulting image reflects the anatomy of the coronary arteries. If the coronary arteries have narrowing as might impede the passage of blood flow to the coronary muscle, the image will reflect the narrowing of the given blood vessel. There is medical value to the prompt and accurate identification of coronary artery narrowing; for example, coronary artery narrowing may inform intervention decisions such as the placement of a coronary artery stent. Current techniques for measuring the amount of coronary artery narrowing rely on visual assessment of the angiographic image and may not be accurate.

### SUMMARY

Embodiments of the invention are directed to methods, systems, and computer readable media for measuring vessels in an angiographic image of a body. Briefly, embodiments of the invention may provide automated, prompt, and accurate measurements of one or more vessels in an angiographic image frame by identifying pixels that correspond to the one or more vessels in the angiographic image frame, locating a medial axis of the one or more vessels, and measuring a dimension transverse to the medial axis of the one or more vessels. The techniques described herein may be applied to any angiographic image of a body, including but not limited to angiographic images obtained at faster than cardiac frequency (e.g., at a frame rate faster than a subject's heart rate) and subjected to spatiotemporal reconstruction (e.g., using wavelet transformations and/or a trained neural network).

In an example embodiment, at least one angiographic image frame is obtained, and pixels corresponding to a vessel in the at least one angiographic frame are identified by assigning each pixel in the angiographic image a probability value (also referred to herein as a “p-value”) as to whether it is a blood vessel or not. In an example embodiment, the p-value is assigned based on an intensity or brightness of the pixel. In an example embodiment, the p-value may range from 0.0 (not a vessel) to 1.0 (a vessel). A probability threshold (e.g., 0.5) may be set for determining whether a pixel represents a blood vessel or not, and the p-value of each pixel may be compared to the threshold to determine whether the pixel represents a blood vessel or not. For example, if the p-value of a pixel exceeds the threshold, the pixel may be determined to represent a vessel. In an example embodiment, each pixel in the image is characterized as either representing a blood vessel or not, and the result of the characterization (i.e., vessel or non-vessel) for each pixel may be stored in a matrix at a specific column and row of the matrix. In an example embodiment, the determination whether a pixel is a vessel or not may be made by a neural network trained to identify pixels corresponding to vessels in an angiographic image. The information from the neural network may be used to generate a angiographic image in which vessels and other structures (such as the angiographic catheter) are segmented. As used herein, the term “to segment” may refer to manually, semi-automatically, or fully-automatically identifying and representing (e.g., displaying) the vascular elements of an angiogram. A vascular “segmentation” may refer to processing of an image such that the vascular structures are represented as distinct from the noise and other structures in the imaged field of view.

In an example embodiment, a medial axis of the one or more vessels may be identified using a medial axis skeletonization technique in which, for every pixel determined to be a vessel, a determination is made whether the pixel is located along a medial axis (i.e., a central longitudinal axis) of the vessel. Pixels determined to be located along the medial axis of a vessel are referred to herein as medial axis pixels. The determination of whether or not a pixel of interest is a medial axis pixel may include drawing or defining a circle (also referred to herein as a “query circle”) around the pixel of interest, and determining whether any pixels intersected by the query circle are non-vessel pixels. If not, the diameter of the query circle is iteratively increased until the circle border includes one or more pixels that are not in the segmented vessel (i.e., one or more non-vessel pixels). If only one non-vessel pixel is intersected by the query circle, then the pixel of interest may be deemed to not be a medial axis pixel. If two non-vessel pixels are intersected by the query circle, then the pixel of interest may be deemed to be located along the medial axis of the vessel (and the diameter of the circle is deemed to be the vessel diameter). If three or more non-vessel pixels are intersected by the query circle, then the pixel of interest may be deemed to be a branch point pixel (e.g., where one vessel branches into two or more vessels). If the query circle fails to intersect any non-vessel pixels, a larger query circle may be drawn around the pixel of interest, and the process may be repeated until at least one of the pixels intersected by the query circle is a non-vessel pixel. In an example embodiment, the first query circle may comprise a group of pixels circumscribing the pixel of interest, and any subsequent query circles may comprise groups of pixels circumscribing previous query circles. A list of all medial axis pixels in the segmented vessel may be generated which corresponds to a list of pixels that are equidistant from a vessel edge. The coordinates of the medial axis pixels (e.g., row and column) may be used to define the medial axis of the vessel, which is essentially a skeletonized representation of the vessel (e.g., in which each segment of the skeleton is only one pixel wide). Additionally, respective diameters of the query circles that intersected two non-vessel pixels around each medial axis pixel may be considered the diameter of the vessel at that point along the medial axis of the vessel. Thus, the medial axis analysis embodiment may produce both the medial axis and diameter of every point in every segmented vessel.

In an example embodiment, a measurement of vessel diameter may be obtained by superimposing the medial axis of the vessel (i.e., the skeleton) onto a segmented vessel image (i.e., an image in which the vascular elements of the angiogram have been identified and represented (e.g., displayed)). The vessel diameter in pixels may be obtained at each medial axis pixel in the skeleton by counting vessel pixels in a transverse direction relative to the medial axis (that is, perpendicular to the medial axis at the medial axis pixel).

In an example embodiment, a pixel size calibration may be performed in order to convert the number of pixels across a width (diameter) of a vessel to units of dimension or size (e.g., millimeters). In an example embodiment, pixel size calibration may be performed by segmenting the angiographic catheter in the angiographic image, performing skeletonization with the medial axis technique, and determining the number of pixels across a width (diameter) of the catheter. The diameter of the catheter is known (e.g., from the catheter manufacturer's FDA package insert) and may be used to determine the image magnification in terms of pixels per unit of size (e.g., pixels per millimeter). The image magnification may then be used as a conversion factor to convert the vessel diameter in pixels to vessel diameter in units of size, such as millimeters.

In one form, a method is provided. The method comprises: obtaining, at a computer, at least one angiographic image frame; identifying, at the computer, pixels corresponding to a vessel of a body in the at least one angiographic image frame; performing, at the computer, an identification of the medial axis of the vessel; and determining, at the computer, a measurement of the vessel in the at least one angiographic image frame in a direction transverse to the medial axis of the vessel.

In one example, identifying the pixels corresponding to the vessel includes: measuring a brightness or intensity of each pixel in the at least one angiographic image frame, classifying the intensity of each pixel as high-intensity or low-intensity based on a threshold, identifying edges in the at least one angiographic image frame based on differences in intensity between adjacent pixels, identifying pixels having a high-intensity signal between two edges as a structure; and identifying the pixels corresponding to the structure as the pixels corresponding to the vessel. For example, the intensity of each pixel may be represented by a floating point number ranging from 0.0 to 1.0.

In another example, identifying the pixels corresponding to the vessel includes assigning each pixel in the at least one angiographic image a probability value (p-value) as to whether it is a blood vessel or not. In one example, the p-value is assigned based on a brightness of the pixel. In one example, a probability threshold (e.g., 0.5) may be set for determining whether a pixel represents a blood vessel or not, and the p-value of each pixel may be compared to the threshold to determine whether the pixel represents a blood vessel or not. In one example, if the p-value of a pixel exceeds the threshold, the pixel may be determined to be part of a vessel.

In a further example, a series of angiographic image frames is obtained that spans at least one cardiac cycle, and the method further comprises: based on the measurement of the vessel in each angiographic image frame of the series of angiographic image frames, determining the measurement of the vessel in the at least one angiographic image frame corresponding to a target time in the at least one cardiac cycle for measuring the vessel.

In one example, identifying the medial axis of a vessel comprises determining, at the computer, which pixels identified as a vessel (vessel pixels) are located along a medial axis of the vessel. In one example, determining whether or not a vessel pixel is located along a medial axis of the vessel includes drawing, at the computer, a query circle around the vessel pixel and determining, at the computer, whether any pixels intersected by the query circle are non-vessel pixels. In one example, if (a) only one non-vessel pixel is intersected by the query circle, then the vessel pixel at the center of the circle is deemed to not be a medial axis pixel; (b) two non-vessel pixels are intersected by the query circle, then the vessel pixel at the center of the circle is deemed to be located along the medial axis of the vessel; or (c) three or more non-vessel pixels are intersected by the query circle, then the vessel pixel at the center of the circle is deemed to be a branch point pixel. In one example, if the query circle fails to intersect any non-vessel pixels, a larger query circle is drawn, at the computer. around the vessel pixel of interest, and the process is repeated until at least one of the pixels intersected by the query circle is a non-vessel pixel.

In one example, identifying the medial axis of the vessel further comprises obtaining, at the computer, a measurement of vessel diameter by superimposing the medial axis of the vessel (i.e., a skeletonized representation of the vessel) onto a version of the angiographic image (which may, for example, be a segmented image such as a spatiotemporally reconstructed version of the angiographic image in which vessels and other structures have been segmented or even an unsegmented image of suitable quality). In one example, obtaining the measurement of vessel diameter includes counting, at the computer, all vessel pixels across a width of the vessel transverse to the medial axis.

In one example, determining a measurement of a vessel in the at least one angiographic image comprises performing, at the computer, a pixel size calibration in order to convert the number of pixels across a width of the vessel to a size dimension. In one example, performing a pixel size calibration includes identifying, at the computer, a catheter in the angiographic image; determining, at the computer, a diameter of the catheter in pixels; retrieving, at the computer, a diameter of the catheter in a unit of size (e.g., millimeters); and determining, at the computer, a conversion factor between pixels and the unit of size. The diameter of the catheter in a unit of size (e.g., millimeters) may then be determined based on the conversion factor between pixels and the unit of size.

In one example, the method further comprises: transforming, at the computer, the at least one angiographic image frame by segmentation of the vessels using a machine learning technique.

In still another example, the method further comprises determining, at the computer, a plurality of diameter measurements along a length of a vessel in the angiographic image. In one example, the method further comprises: rendering a heat map that indicates the plurality of measurements of the vessel in the at least one angiographic image frame.

In another example, the method further comprises comparing, at the computer, the diameter of at least one portion of a vessel in the at least one angiographic image frame to a reference measurement of the vessel. In an additional example, the reference measurement is a diameter of a second portion of the vessel located upstream of the at least one vessel portion. In yet an additional example, the method further comprises displaying, at the computer, a visual indication of stenosis adjacent the at least one portion of a vessel if the measured diameter of the at least one portion of the vessel is less than a predetermined percentage of the diameter of the second portion of the vessel. For example, a common guideline for determining stenosis from the American Heart Association is 70% or greater stenosis, which is when the vessel diameter is 30% or less than a prior diameter of the vessel subsequent to the preceding branch point.

In yet another example, the method further comprises displaying, at the computer, a graphical user interface showing one or more icons representing stents. In one example, the one or more stent icons are selected for display, at the computer, based on their suitability for treating a stenosis indicated in the at least one angiographic image frame and/or their availability in inventory. In one example, the graphical user interface displays information about the one or more stents. In still another example, the method further comprises moving, at the computer, a selected stent icon onto the angiographic image in response to a user input. In a further example, the method further comprises displaying, at the computer, the selected stent icon in a correct proportion relative to a vessel segment on which it is placed. In yet a further example, the method further comprises receiving, at the computer, a user selection of a stent; and, sending, at the computer, information about the selected stent to an inventory and/or billing system.

In yet another example, the method further comprises obtaining, at the computer, a plurality of angiographic images of a body at a frame rate that is faster than a cardiac frequency (i.e., heart rate) of the patient, and performing a spatiotemporal reconstruction of the angiographic images. In further examples, the spatiotemporal reconstruction may be performed, at the computer, using a wavelet transformation (e.g., as described in U.S. application Ser. No. 16/784,073, filed on Feb. 6, 2020, which is hereby incorporated by reference in its entirety) and/or a neural network trained to detect spatiotemporal properties of contrast-containing vascular structures. For example, the neural network may be trained to estimate the vascular structures in a given image from the target image and from one or more temporally adjacent images.

In another form, a system is provided. The system comprises: a communications interface configured to obtain at least one angiographic image frame; and one or more processors coupled to the communications interface, wherein the one or more processors are configured to: identify pixels corresponding to a vessel of a body in the at least one angiographic image frame; identify a medial axis of the vessel; and determine a measurement of the vessel in the at least one angiographic image frame in a direction transverse to the medial axis of the vessel.

In another form, one or more non-transitory computer readable storage media are provided. The one or more non-transitory computer readable storage media are encoded with instructions that, when executed by a processor, cause the processor to: obtain at least one angiographic image frame; identify pixels corresponding to a vessel of a body in the at least one angiographic image frame; identify a medial axis of the vessel; and determine a measurement of the vessel in the at least one angiographic image frame in a direction transverse to the medial axis of the vessel.

Still other objects and advantages of these techniques will be apparent from the specification and drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A and 1B are side and partially schematic views, respectively, showing an example of a rotational x-ray system that may be used with embodiments of the disclosure for acquiring angiographic data.

FIG. 2 is a schematic diagram of a computer system or information processing device that may be used with embodiments of the disclosure.

FIG. 3 is a perspective view of a pulse oximeter coupled to a multi-parameter patient monitor and a sensor that may be used with aspects of the disclosure for acquiring a cardiac signal, according to an example embodiment of the disclosure.

FIG. 4 is a block diagram of an electrocardiogram (EKG) device that may be used with aspects of the disclosure for acquiring a cardiac signal, according to an example embodiment of the disclosure.

FIG. 5A illustrates an angiographic image showing a plurality of vessels in a body, according to an example embodiment of the disclosure.

FIG. 5B illustrates a segmented angiographic image derived from the angiographic image of FIG. 5A, according to an example embodiment of the disclosure.

FIG. 5C illustrates a method for identifying the medial axis of a vessel whereby pixels corresponding to a vessel are determined to be located along a medial axis of the vessel, or not located along the medial axis, or located adjacent a branch point of the vessel, according to an example embodiment of the disclosure.

FIG. 5D illustrates a skeletonized angiographic image derived from the segmented angiographic image of FIG. 5B using a medial axis skeletonization method, according to an example embodiment of the disclosure.

FIG. 5E illustrates a superposition of the skeletonized angiographic image of FIG. 5C on the segmented angiographic image of FIG. 5B, according to an example embodiment of the disclosure.

FIG. 5F illustrates segmentation of a catheter in an angiographic image, according to an example embodiment of the disclosure.

FIG. 5G illustrates a heat map indicative of the size of the catheter in the angiographic image, according to an example embodiment of the disclosure.

FIG. 5H illustrates a heat map indicative of a measurement of the size of a vessel of a body, according to an example embodiment of the disclosure.

FIG. 6 illustrates a graphical user interface for an angiographic system, according to an example embodiment of the disclosure.

FIG. 7 is a flowchart showing a method for measuring vessels in a body, according to an example embodiment of the disclosure.

FIG. 8 is a flowchart showing a method for identifying pixels corresponding to a structure (such as a vessel of a body or a catheter) in at least one angiographic image frame, according to an example embodiment of the disclosure.

FIG. 9 is a flowchart showing a method for identifying the medial axis of a vessel of a body, according to an example embodiment of the disclosure.

FIG. 10 is a flowchart showing a method for measuring the diameter of a vessel in the body, according to an example embodiment of the disclosure.

FIG. 11 is a flowchart showing a method for converting a diameter in pixels to a diameter in units of size, according to an example embodiment of the disclosure.

### DETAILED DESCRIPTION OF EXAMPLE EMBODIMENTS

Techniques are described herein for measuring a vessel (e.g., a blood vessel) in a body. The vessel may be an artery or a vein, and may have any suitable location within the body. In one specific example, the vessel may be a coronary artery. The body may be a mammalian body, such as a human body. In one example, the vessel may be captured in an angiographic image frame, the angiographic image may be segmented, a medial axis of the vessel may be identified, and a measurement of the vessel may be obtained by measuring a dimension transverse to the medial axis of the vessel.

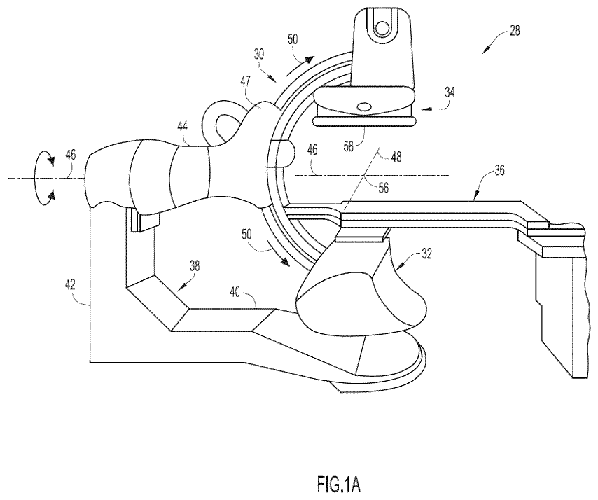

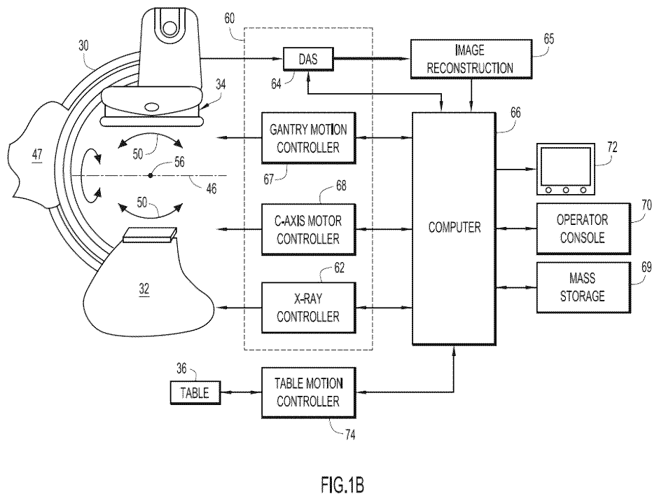

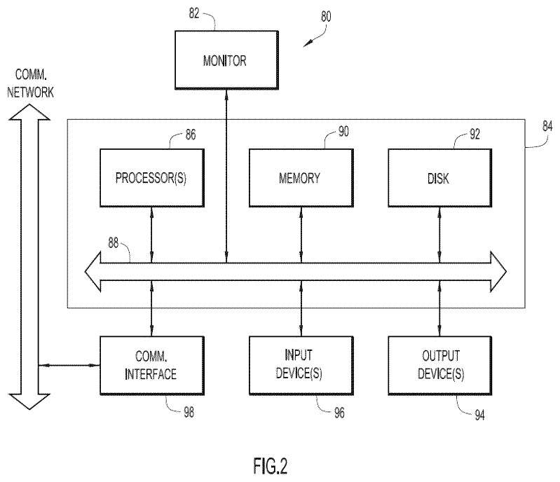

Referring to FIGS. 1A, 1B, and 2, exemplary systems or devices that may be employed for carrying out embodiments of the invention are illustrated. It is understood that such systems and devices are only exemplary of representative systems and devices and that other hardware and software configurations are suitable for use with embodiments of the invention. Thus, the embodiments are not intended to be limited to the specific systems and devices illustrated herein, and it is recognized that other suitable systems and devices can be employed without departing from the spirit and scope of the subject matter provided herein.

Referring first to FIGS. 1A and 1B, a rotational x-ray system 28 is illustrated that may be employed for obtaining an angiogram at faster than cardiac rate, such as via fluoroscopic angiography. In acquiring an angiogram, a chemical contrast agent may be injected into the patient positioned between an x-ray source and detector, and x-ray projections are captured by the x-ray detector as a two-dimensional image (i.e., an angiographic image frame). A sequence of such image frames comprises an angiographic study, and, in some embodiments of the invention, the angiographic image frames may be acquired at faster than cardiac frequency to facilitate vessel measurement.

As shown in FIG. 1A, an example of an angiogram imaging system is shown in the form of a rotational x-ray system 28 including a gantry having a C-arm 30 which carries an x-ray source assembly 32 on one of its ends and an x-ray detector array assembly 34 at its other end. The gantry enables the x-ray source assembly 32 and x-ray detector array assembly 34 to be oriented in different positions and angles around a patient disposed on a table 36, while providing to a physician access to the patient. The gantry includes a pedestal 38 which has a horizontal leg 40 that extends beneath the table 36 and a vertical leg 42 that extends upward at the end of the horizontal leg 40 that is spaced apart from table 36. A support arm 44 is rotatably fastened to the upper end of vertical leg 42 for rotation about a horizontal pivot axis 46.

The horizontal pivot axis 46 is aligned with the centerline of the table 36, and the support arm 44 extends radially outward from the horizontal pivot axis 46 to support a C-arm drive assembly 47 on its outer end. The C-arm 30 is slidably fastened to the C-arm drive assembly 47 and is coupled to a drive motor (not shown) which slides the C-arm 30 to revolve about a C-axis 48 as indicated by arrows 50. The horizontal pivot axis 46 and C-axis 48 intersect each other, at a system isocenter 56 located above the table 36, and are perpendicular to each other.

The x-ray source assembly 32 is mounted to one end of the C-arm 30 and the x-ray detector array assembly 34 is mounted to its other end. The x-ray source assembly 32 emits a beam of x-rays which are directed at the x-ray detector array assembly 34. Both assemblies 32 and 34 extend radially inward to the horizontal pivot axis 46 such that the center ray of this beam passes through the system isocenter 56. The center ray of the beam thus can be rotated about the system isocenter around either the horizontal pivot axis 46 or the C-axis 48, or both, during the acquisition of x-ray attenuation data from a subject placed on the table 36.

The x-ray source assembly 32 contains an x-ray source which emits a beam of x-rays when energized. The center ray passes through the system isocenter 56 and impinges on a two-dimensional flat panel digital detector 58 housed in the x-ray detector array assembly 34. The two-dimensional flat panel digital detector 58 may be, for example, a 2048×2048 element two-dimensional array of detector elements. Each element produces an electrical signal that represents the intensity of an impinging x-ray and hence the attenuation of the x-ray as it passes through the patient. During a scan, the x-ray source assembly 32 and x-ray detector array assembly 34 are rotated about the system isocenter 56 to acquire x-ray attenuation projection data from different angles. In example devices, the detector array may be able to acquire a given number of (e.g., up to fifty) projections, or image frames, per second. The rate of image frames per second may determine how many image frames can be acquired for a prescribed scan path and speed.

Referring to FIG. 1B, the rotation of the assemblies 32 and 34 and the operation of the x-ray source are governed by a control mechanism 60 of the x-ray system. The control mechanism 60 includes an x-ray controller 62 that provides power and timing signals to the x-ray source assembly 32. A data acquisition system (DAS) 64 in the control mechanism 60 samples data from detector elements and passes the data to an image reconstructor 65. The image reconstructor 65 receives digitized x-ray data from the DAS 64 and performs high speed image reconstruction according to the methods of the present disclosure. The reconstructed image is applied as an input to a computer 66 which stores the image in a mass storage device 69 or processes the image further. Image reconstructor 65 may be a standalone computer or may be integrated with computer 66.

The control mechanism 60 also includes gantry motor controller 67 and a C-axis motor controller 68. In response to motion commands from the computer 66, the motor controllers 67 and 68 provide power to motors in the x-ray system that produce the rotations about horizontal pivot axis 46 and C-axis 48, respectively. The computer 66 also receives commands and scanning parameters from an operator via console 70 that has a keyboard and other manually operable controls. An associated display 72 allows the operator to observe the reconstructed image frames and other data from the computer 66. The operator supplied commands are used by the computer 66 under the direction of stored programs to provide control signals and information to the DAS 64, the x-ray controller 62 and the motor controllers 67 and 68. In addition, computer 66 operates a table motor controller 74 which controls the motorized table 36 to position the patient with respect to the system isocenter 56.

Referring now to FIG. 2, a block diagram of a computer system or information processing device 80 (e.g., image reconstructor 65 and/or computer 66 in FIG. 1B) is illustrated that may be incorporated into an angiographic imaging system, such as the rotational x-ray system 28 of FIGS. 1A and 1B, to provide enhanced functionality or to be used as a standalone device for vessel measurement and extraction of cardiac frequency phenomena from angiographic data according to an embodiment of the present invention. Information processing device 80 may be local to or remote from rotational x-ray system 28. In one example, the functionality performed by information processing device 80 may be offered as a Software-as-a-Service (SaaS) option. SaaS refers to a software application that is stored in one or more remote servers (e.g., in the cloud) and provides one or more services (e.g., angiographic image processing) to remote users. In one embodiment, computer system 80 includes monitor or display 82, computer system 84 (which includes processor(s) 86, bus subsystem 88, memory subsystem 90, and disk subsystem 92), user output devices 94, user input devices 96, and communications interface 98. Monitor 82 can include hardware and/or software elements configured to generate visual representations or displays of information. Some examples of monitor 82 may include familiar display devices, such as a television monitor, a cathode ray tube (CRT), a liquid crystal display (LCD), or the like. In some embodiments, monitor 82 may provide an input interface, such as incorporating touch screen technologies.

Computer system 84 can include familiar computer components, such as one or more central processing units (CPUs), memories or storage devices, graphics processing units (GPUs), communication systems, interface cards, or the like. As shown in FIG. 2, computer system 84 may include one or more processor(s) 86 that communicate with a number of peripheral devices via bus subsystem 88. Processor(s) 86 may include commercially available central processing units or the like. Bus subsystem 88 can include mechanisms for letting the various components and subsystems of computer system 84 communicate with each other as intended. Although bus subsystem 88 is shown schematically as a single bus, alternative embodiments of the bus subsystem may utilize multiple bus subsystems. Peripheral devices that communicate with processor(s) 86 may include memory subsystem 90, disk subsystem 92, user output devices 94, user input devices 96, communications interface 98, or the like.

Processor(s) 86 may be implemented using one or more analog and/or digital electrical or electronic components, and may include a microprocessor, a microcontroller, an application-specific integrated circuit (ASIC), a field programmable gate array (FPGA), programmable logic and/or other analog and/or digital circuit elements configured to perform various functions described herein, such as by executing instructions stored in memory subsystem 90 and/or disk subsystem 92 or another computer program product.

Memory subsystem 90 and disk subsystem 92 are examples of physical (non-transitory) storage media configured to store data. Memory subsystem 90 may include a number of memories including random access memory (RAM) for volatile storage of program code, instructions, and data during program execution and read only memory (ROM) in which fixed program code, instructions, and data are stored. Disk subsystem 92 may include a number of file storage systems providing persistent (non-volatile) storage for programs and data. Other types of physical storage media include floppy disks, removable hard disks, optical storage media such as compact disc-read-only memories (CD-ROMS), digital video disc (DVDs) and bar codes, semiconductor memories such as flash memories, read-only-memories (ROMS), battery-backed volatile memories, networked storage devices, or the like. Memory subsystem 90 and disk subsystem 92 may be configured to store programming and data constructs that provide functionality or features of techniques discussed herein. Software code modules and/or processor instructions that when executed by processor(s) 86 implement or otherwise provide the functionality may be stored in memory subsystem 90 and disk subsystem 92. Memory, subsystem 90 may be a non-transitory computer readable storage medium.

User input devices 96 can include hardware and/or software elements configured to receive input from a user for processing by components of computer system 80. User input devices can include all possible types of devices and mechanisms for inputting information to computer system 84. These may include a keyboard, a keypad, a touch screen, a touch interface incorporated into a display, audio input devices such as microphones and voice recognition systems, and/or other types of input devices. In various embodiments, user input devices 96 may include a computer mouse, a trackball, a track pad, a joystick, a wireless remote, a drawing tablet, a voice command system, an eye tracking system, or the like. In some embodiments, user input devices 96 are configured to allow a user to select or otherwise interact with objects, icons, text, or the like that may appear on monitor 82 via a command, motions, or gestures, such as a click of a button or the like.

User output devices 94 can include hardware and/or software elements configured to output information to a user from components of computer system 80. User output devices can include all possible types of devices and mechanisms for outputting information from computer system 84. These may include a display (e.g., monitor 82), a printer, a touch or force-feedback device, audio output devices, or the like.

Communications interface 98 can include hardware and/or software elements configured to provide unidirectional or bidirectional communication with other devices. For example, communications interface 98 may provide an interface between computer system 84 and other communication networks and devices, such as via an internet connection.

In one example, information processing device 80 may obtain (e.g., via communications interface 98) a series of angiographic image frames (e.g., an angiogram) at a rate faster than cardiac frequency. For instance, information processing device 80 may use wavelet angiography (e.g., by employing complex-valued wavelet transforms) to generate a spatiotemporal reconstruction of cardiac frequency phenomena in an angiogram obtained at faster than cardiac frequency. Spatiotemporal reconstruction may be performed in accordance with techniques described in U.S. Pat. No. 10,123,761, issued Nov. 13, 2018, which is hereby incorporated by reference in its entirety.

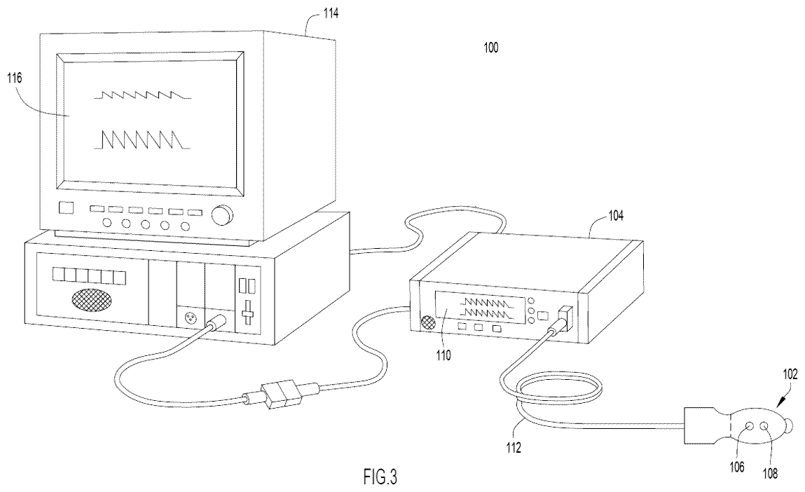

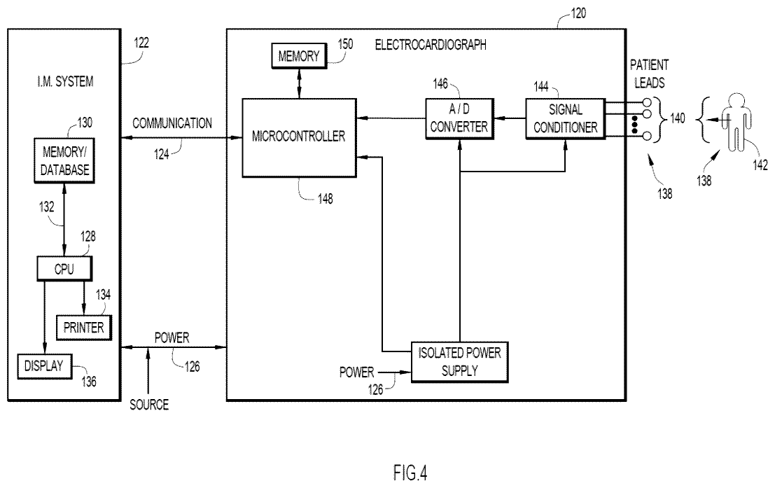

In addition to acquiring angiographic images, additional cardiac signals/data may be contemporaneously acquired to serve as a cross correlation target. For example, the cardiac signals/data may serve as a reference cardiac signal for phase indexing pixels in the angiographic projections. FIGS. 3 and 4 illustrate exemplary devices for acquiring/providing a reference cardiac signal with such devices/systems in the form of a pulse oximetry system and/or an echocardiogram (EKG) system or device.

FIG. 3 is a perspective view of an example of a suitable pulse oximetry system 100 that includes a sensor 102 and a pulse oximetry monitor 104. The sensor 102 includes an emitter 106 for emitting light at certain wavelengths into a patient's tissue and a detector 108 for detecting the light after it is reflected and/or absorbed by the patient's tissue. The monitor 104 may be capable of calculating physiological characteristics received from the sensor 102 relating to light emission and detection. Further, the monitor 104 includes a display 110 capable of displaying the physiological characteristics and/or other information about the system. The sensor 102 is shown communicatively coupled to the monitor 104 via a cable 112, but alternatively may be communicatively coupled via a wireless transmission device or the like.

In the illustrated embodiment the pulse oximetry system 100 also includes a multi-parameter patient monitor 114. In addition to the monitor 104, or alternatively, the multi-parameter patient monitor 114 may be capable of calculating physiological characteristics and providing a central display 116 for information from the monitor 104 and from other medical monitoring devices or systems. For example, the multi-parameter patient monitor 114 may display a patient's SpO2 and pulse rate information from the monitor 104 and blood pressure from a blood pressure monitor on the display 116.

In an embodiment, computer system 80 may be configured to include hardware and software for communicating with a pulse oximetry sensor, such as the sensor 102 shown in FIG. 3, as well as hardware and software to calculate physiological characteristics received from the pulse oximetry sensor and utilize such characteristics to extract cardiac frequency phenomena, and display the same, in accordance with the techniques described herein.

FIG. 4 is a schematic diagram of an electrocardiogram (“EKG”) device 120 shown optionally connected to an information management system 122 through a communications link 124. A commonly used device for acquiring an EKG is a 12-lead electrocardiograph. The EKG device 120 and the information management system 122 receives power 126 from an external source. Among other things, the information management system 122 includes a central processing unit 128 connected to a memory unit, or database 130, via a data link 132. The CPU 128 processes data and is connected to an output, such as printer 134 and/or display 136. Alternatively, the electrocardiogram (EKG) device 120 can be connected directly to a printer 134 or display 136 through communications link 124, if the optional information management system 122 is not utilized.

In an embodiment, the software program according to embodiments provided herein may reside in the EKG device 120, the information management system 122, or another device configured to receive signals from the EKG device 120. The EKG device 120 is connected to a plurality of patient lead wires 138, each having an electrode 140 to receive EKG signals from a patient 142. The EKG device 120 has a signal conditioner 144 that receives the EKG signals and filters noise, sets thresholds, segregates signals, and provides the appropriate number of EKG signals for the number of leads 138 to an A/D converter 146 which converts the analog signals to digital signals for processing by a microcontroller 148, or any other type of processing unit. Microcontroller 148 is connected to a memory unit 150, similar to memory unit 130, or any other computer readable storage medium.

In another embodiment, computer system 80 may be configured to include hardware and software for communicating with an EKG device, such as EKG device 120, or to perform the functions of an EKG device by communicating directly with EKG electrodes, such as electrodes 140 shown in FIG. 4, and calculating physiological characteristics received from the electrodes and utilizing such characteristics to extract and display cardiac frequency phenomena.

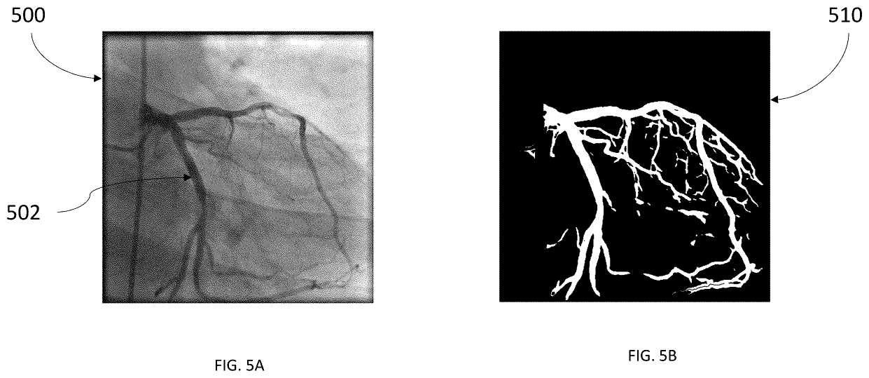

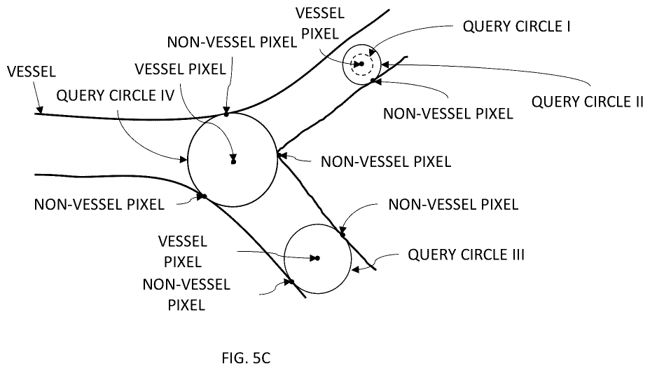

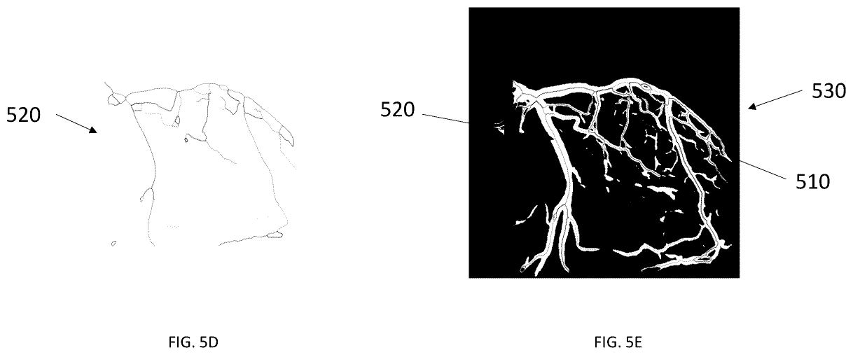

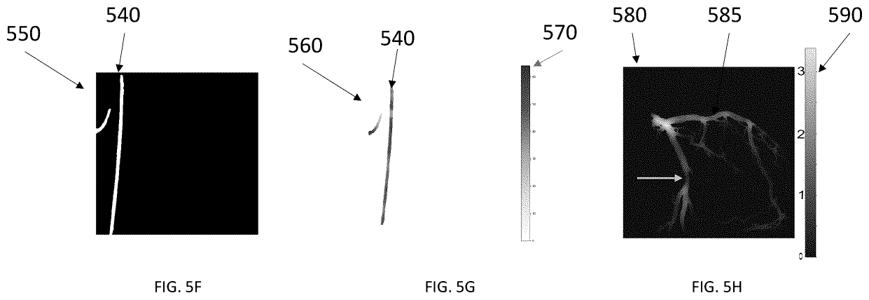

FIG. 5A illustrates an example of an angiographic image frame 500 that may be obtained, for example, by injecting a contrast agent into a blood vessel of a subject and imaging the body of the subject with a rotational x-ray system of the type shown in FIGS. 1A and 1B. A plurality of vessels 502 of the body are shown in the angiographic image frame 500.

In one example, a computer (e.g., comprising image reconstructor 65, computer 66, and/or computer system 80) may obtain angiographic image frame 500, e.g., via rotational x-ray system 28. The computer may further identify pixels corresponding to a vessel of a body in angiographic image frame 500. For example, the computer may measure the brightness or intensity of each pixel in angiographic image frame 500 and assign each pixel in the at least one angiographic image a probability value (p-value) as to whether it is a blood vessel or not. In one example, the p-value is assigned based on a brightness or intensity of the pixel. In one example, a binarization threshold may be set for determining whether a pixel represents a blood vessel or not, and the p-value of each pixel may be compared to the binarization threshold to determine whether the pixel represents a blood vessel or not. In one example, if the p-value of a pixel exceeds the threshold, the pixel may be determined to correspond to a vessel. It has been found that p-values may jump abruptly (e.g., from 0.1 to 0.9) between pixels that are not vessels and pixels that are vessels. Therefore, the binarizaton threshold may be set anywhere in that range; however, consistently good results have been recorded using a binarization threshold of 0.5. In one embodiment, an encoder-decoder neural network may be trained to assign a p-value to every pixel as to whether it corresponds to a vessel or not. The neural network may also be used to identify pixels corresponding to other structures in the angiographic image frame, such as a catheter.

FIG. 5B illustrates a segmented angiographic image 510 derived from the angiographic image frame 500 shown in FIG. 5A, according to an example embodiment of the disclosure. In this example, the segmented angiographic image 510 was generated by a neural network (e.g., having an input layer, an output layer, and three or more layers between the input and output layers) trained to identify vessels in an angiographic image frame. While descriptions provided herein will focus on deep learning neural networks, it will be appreciated that these techniques may be utilized with any suitable machine learning model, of which a deep learning neural network is just one example. The machine learning model may be implemented by any suitable machine learning techniques (e.g., mathematical/statistical, classifiers, feed-forward, recurrent, convolutional, or other neural networks, etc.). For example, a neural network may be used that includes an input layer, one or more intermediate layers (e.g., including any hidden layers), and an output layer. Each layer may include one or more nodes or neurons, where the input layer neurons receive input (e.g., image data, feature vectors of images, etc.), and may be associated with weight values. The neurons of the intermediate and output layers may be connected to one or more neurons of a preceding layer, and receive as input the output of a connected neuron of the preceding layer. Each connection may be associated with a weight value, and each neuron may produce an output based on a weighted combination of the inputs to that neuron. The output of a neuron may further be based on a bias value for certain types of neural networks (e.g., recurrent types of neural networks). The weight (and bias) values may be adjusted based on various training techniques. For example, the machine learning of the neural network may be performed using a training set of raw angiographic images as input and corresponding manually segmented angiographic images as known outputs, where the neural network attempts to produce the provided output and uses an error from the output (e.g., difference between produced and known outputs) to adjust weight (and bias) values (e.g., via backpropagation or other training techniques).

The training data may be acquired based on any suitable angiographic training images. For example, the training data may be obtained (1) in a laboratory setting using animals undergoing approved angiographic studies; (2) in a laboratory setting using physical models with fluid mechanically pumped into synthetic organs which are angiographically imaged; and/or (3) from human clinical angiographic data. The data training system may use these training components alone or in any suitable combination to train the deep learning neural network.

The neural network techniques described herein may be utilized in combination with spatiotemporal reconstruction techniques as described in U.S. application Ser. No. 16/784,073, filed on Feb. 6, 2020, which is hereby incorporated by reference in its entirety. The spatiotemporal reconstruction of an image may be an input to the neural network, or the output of the neural network may be processed using spatiotemporal reconstruction.

The computer may analyze all pixels in angiographic image frame 500 or a subset of pixels in angiographic image frame 500. For example, a user (e.g., a medical professional) may select a subset of pixels within angiographic image frame 500 and the computer may perform the measurement analysis on pixels within the selected subset that correspond to one or more vessels.

The computer may analyze the pixels corresponding to a vessel in angiographic image frame 500 to identify a medial axis of the vessel and, based on the medial axis analysis, determine a measurement of the vessel. The computer may further display a scale to indicate a measurement (e.g., length). The scale may, for example, be automatically superimposed on angiographic image frame 500 (e.g., on the measured vessel).

In some embodiments, the medial axis analysis may provide measurements of multiple (e.g., all) blood vessels in angiographic image frame 500. For example, the computer may identify pixels corresponding to a plurality of vessels of the body in angiographic image frame 500. The computer may perform the medial axis analysis on the pixels corresponding to the plurality of vessels. Based on the medial axis analysis, the computer may determine measurements (e.g., diameters) of the plurality of vessels in angiographic image frame 500.

In one example, the computer may compare the measurement of the vessel in angiographic image frame 500 (e.g., a measured diameter of the vessel) to a reference measurement of the vessel (e.g., a reference diameter of the vessel). Based on the comparison, the computer may calibrate and/or validate the medial axis analysis. The reference diameter may be obtained via an angiographic injection catheter method. For example, a neural network may identify an injection catheter in angiographic image frame 500. The diameter of the injection catheter may be known (e.g., determined from a catalog or package insert) and used to determine the vessel diameter. For instance, if an injection catheter has a diameter of 6 French units, then the diameter is 2 mm (3 French units are defined to be equal to 1 mm). The measurement obtained from the diameter of the injection catheter may provide validation of the diameter measurement(s) obtained from angiographic image frame 500.

In an example embodiment, a series/sequence of angiographic image frames that span a cardiac cycle may be obtained. The series of angiographic image frames may be obtained from an angiogram in temporal sequence according to the duration of the presence of the bolus of contrast in the organ being imaged. In one example, a computer may identify pixels corresponding to the vessel in each angiographic image frame. The computer may perform the medial axis analysis on the pixels corresponding to the vessel in each angiographic image frame. Based on the medial axis analysis, the computer may determine the measurement of the vessel in each angiographic image frame.

In a further example, the series of angiographic image frames spans at least one cardiac cycle. Based on the measurement of the vessel in each angiographic image frame, the computer may determine that the measurement of the vessel in at least one angiographic image frame was determined at a target/preferred time in the cardiac cycle for measuring the vessel. As a result, the computer may select the target time in the cardiac cycle at which to measure the diameter of the blood vessel.

Consider an example in which a heart is being imaged. The heart contracts at systole and relaxes at diastole. By repeating the blood vessel automated measurements for all images across the cardiac cycle, the computer may determine the vessel diameter as it varies across the cardiac cycle. The computer may further select the preferred/target time point in the cardiac cycle that has the greatest value for clinical decision-making for vessel measurement. In coronary angiography, the preferred time point for coronary artery diameter measurement may correspond to end-diastole.

The application of automated vessel measurements to all images in a coronary angiogram may produce a spatio-temporal family of vessel diameter measurements across every cardiac cycle in the travel of a contrast bolus in a coronary angiogram. The resulting measurements may help improve decisions regarding treatment, such as whether to place a coronary artery stent and, if so, the appropriate size of the stent.

FIG. 5C illustrates an example embodiment of a method for determining whether pixels corresponding to a vessel (also referred to as “vessel pixels”) are located along a medial axis (i.e., central longitudinal axis) of the vessel or not. Vessel pixels located along the medial axis of a vessel are also referred to herein as a “medial axis pixel.” In this example, the medial axis analysis technique may also be used to determine whether or not a vessel pixel is located adjacent a branch point of the vessel.

Briefly, for every pixel determined to be a vessel pixel (e.g., by the binarization technique described above), the medial axis analysis technique draws a tiny circle (also referred to as a “query circle”) around the vessel circle and determines whether or not the circle intersects one or more pixels that do not correspond to a vessel (also referred to as “non-vessel pixels”). The size of the query circle may be increased iteratively if the previous query circle did not intersect at least one non-vessel pixel, until the query circle intersects at least one non-vessel pixel. In an example embodiment, the first query circle may comprise a group of pixels circumscribing the pixel of interest, and any subsequent query circles may comprise groups of pixels circumscribing previous query circles.

If the first query circle fails to intersect a non-vessel pixel (see, e.g., query circle 1 in FIG. 5C), query circles of larger diameter are drawn (see, e.g., query circle II in FIG. 5C) iteratively until at least one non-vessel pixel is intersected. If, at a certain diameter, the query circle intersects a single non-vessel pixel (see, e.g., query circle II in FIG. 5C), the vessel pixel at the center of the query circle is considered to not be a medial axis pixel. If at a certain diameter, the query circle intersects two non-vessel pixels (see, e.g., query circle III in FIG. 5C), the vessel pixel at the center of the query circle is considered to be a medial axis pixel (i.e., located along the medial axis, at a center of the vessel, equidistant from opposite edges of the vessel). If at a certain diameter, the query circle intersects three or more non-vessel pixels (see, e.g., query circle IV in FIG. 5C), the vessel pixel at the center of the query circle is considered to be located at a branch point of the vessel (e.g., where one vessel segment branches into two vessel segments).

An example embodiment of the technique may be expressed as follows:

```
For every row:
  For every column:
    If a pixel at (row, column) is segmented as vessel:
      Draw circles of increasing diameter around pixel:
        If a given diameter hits only one non-segmented (i.e.,
      non-vessel) pixel:
          Move on to next (row, column).
        If a given diameter hits two non-segmented pixels:
          Record (row, column) as vessel medial axis;
          Record circle diameter as vessel diameter; and
          Move on to next (row, column).
        If a given diameter hits three or more non-segmented
      pixels:
          Record (row, column) as branch location;
          Record circle diameter as branch diameter; and
        Move on to next (row, column).
```

In an example embodiment, the technique may record a list of all pixels in the segmented vessel that are equidistant from a vessel edge. That is, the coordinates (e.g., row, column) of every medial axis pixel may be recorded. The list of coordinates defines the medial axis of the segmented vessel, which may also be thought of as a skeletonized representation of the segmented vessel. (In an example embodiment, the skeleton may be one pixel wide.) There are several ways to assess medial axis points adjacency. One is from the sequence of the row, column iteration. Another is from analysis of the medial axis coordinates themselves. The technique works on binarized, pixelated data. The natural resolution is the pixel resolution.

In an example embodiment, the technique may determine a width of a vessel in an angiographic image frame (which may be considered to correspond to a diameter of the vessel in real life) at each point (i.e., medial axis pixel) along the medial axis of the vessel. The width is preferably determined in a transverse (i.e., perpendicular) direction relative to the medial axis of the vessel. The transverse direction may be calculated naturally from the shape of the expanding query circle in the skeletonization technique described above. That is, a measurement of width in a transverse direction may be achieved by drawing query circles of increasing diameter until two or more non-vessel pixels are intersected, and recording the diameter of the query circle that intersects two non-vessel pixels as the diameter of the vessel at that point along the medial axis. Similarly, the diameter of a query circle that intersects three or more non-vessel pixels may be recorded as the diameter of a branch. (In an example embodiment, the diameter of the query circle that intersects two or more non-vessel pixels may be recorded as a number of pixels.) Therefore, the technique may provide the medial axis and diameter of every point in every segmented vessel in an angiographic image frame.

FIG. 5D illustrates an example of a skeletonized representation 520 of the segmented angiographic image 510 of FIG. 5B obtained using the technique described above. Each skeleton segment is only one pixel wide in this example. Since the technique is capable of detecting branch points, it allows segment vessels between branch points to be identified. Segmented vessels are labeled here with an alternating color pallet.

FIG. 5E illustrates how, according to an example embodiment of the present technique, vessel diameter may be measured by superimposing the skeletonized representation 520 of FIG. 5D on the segmented angiographic image 510 of FIG. 5B. The resulting superimposed image is labeled 530 in FIG. 5E. In this example, white pixels are vessel pixels, black pixels are non-vessel pixels, and segments of the skeletonized representation are shown in alternating colors. It can be seen that the skeletonized representation 520 and segmented angiographic image 510 are superimposed so that the segments of the skeleton lie along the medial axis of each vessel. Briefly, for each skeletal segment, iterating from one end of the segment to the other, and at each pixel in the skeleton, a diameter of the vessel is measured in pixels by counting how many pixels it takes in a transverse direction to get to a black pixel. For example, the number of pixels in a transverse direction from the medial axis pixel to a black pixel (i.e., the radius of the vessel) may be counted and doubled to obtain the diameter. Alternatively, the number of pixels in a transverse direction corresponding to the diameter (i.e., the number of white pixels between black pixels in the transverse direction) may be counted.

A pixel size calibration may be performed in order to convert the number of pixels across a width (diameter) of a vessel to units of size or dimension (e.g., millimeters). In an example embodiment, pixel size calibration may be performed by segmenting an angiographic catheter in the angiographic image (see, e.g., FIG. 5F), performing skeletonization with the medial axis technique, and determining the number of pixels across a width (diameter) of the catheter. The diameter of the catheter is known (e.g., from the catheter manufacturer's FDA package insert) and may be used to determine the image magnification in terms of pixels per unit of size (e.g., pixels per millimeter). The image magnification (or its inverse) may then be used as a conversion factor to convert the vessel diameter in pixels to vessel diameter in units of size, such as millimeters.

FIG. 5F illustrates segmentation of a catheter 540 in an angiographic image 550, according to an example embodiment of the disclosure. In this illustration, the catheter 540 is displayed as a white line. Segmentation of the catheter 540 may be performed using a neural network, such as the neural network described above, trained using raw angiographic images of a catheter in a body as inputs and manually segmented images of the catheter in the body as a known output, for example.

FIG. 5G illustrates a heat map 560 indicative of the diameter of the catheter 540 in pixels. In one example, the computer may render heat map 560. The heat map 560 includes a color image of the catheter 540 on the left, in which the color and hue of the catheter in the image is indicative of its size, and an image legend or scale 570 to the right of the image equating colors/hues in the image to a number of pixels. As noted above, the catheter diameter may be looked up in the catheter manufacturer's FDA package insert and inputted into the computer. Alternatively, the catheter diameter may be retrieved by the computer from a database. In the example shown, the catheter is known to have a 2 mm diameter. The image magnification in terms of pixels per millimeter may be calculated by dividing the size of the catheter in pixels by the known diameter in millimeters. For example, in the case of a 2 mm diameter catheter, if the heat map indicates that the catheter is 60 pixels wide (i.e., has a diameter of 60 pixels), the image magnification would be 30 pixels per millimeter, and the conversion factor for millimeters per pixel would be 1 millimeter per 30 pixels. Of course, units of size other than millimeters can be used.

As noted above, the conversion factor determined by the computer based on the known diameter of the catheter and the number of pixels measured transversely across the width of the catheter in the angiographic image, can be used by the computer to convert vessel diameter in pixels to vessel diameter in units of size, such as millimeters. For example, if the conversion factor is 1 millimeter per 30 pixels, and the vessel diameter at a particular point along its medial axis is measured as 30 pixels, the diameter of the vessel in units of size at that point would be 1 millimeter (i.e., 30 pixels×1 mm/30 pixels=1 mm). In this way, the diameter of every point in every segmented vessel in an angiographic image frame may be automatically measured.

FIG. 5H illustrates a heat map 580 indicative of a measurement of a vessel of a body, according to an example embodiment of the disclosure. In one example, the computer may render heat map 580. For example, the computer may display the heat map 580 on a monitor or other type of screen. The computer may also store the heat map 580 as a digital image, which may (among other things) be retrieved by the computer at any time, copied to a transportable computer readable storage medium such as a USB thumbdrive or a DVD, transmitted to another computer over an electronic communications network, or transmitted to a printer for printing. In the example shown, the heat map 580 includes a color image 585 of the vessels on the left, in which the color and hue of the vessels in the image is indicative of their diameter, and an image legend, scale, or key 590 to the right of the image correlating colors/hues in the image to dimensions and/or dimensional ranges in units of size (e.g., millimeters). Using the heatmap, the diameter of a vessel at any point along its length can be determined by medical professionals by comparing the color and hue of the vessel at that point with the legend, scale, or key 590.

The vessel diameter data may be screened for evidence of local stenosis as might meet criteria for the placement of a coronary artery stent. The contemporary criteria are listed in the 2021 ACC/AHA/SCAI Guideline for Coronary Artery Revascularization, available in Section 4.1 of https://www.jacc.org/doi/10.1.016/j.jacc.2021.09.006. These criteria are a left main coronary artery stenosis of 50% or greater, or stenosis of 70% or greater in non-left main coronary artery stenosis. The guidelines state this may be visually estimated, but do not give a quantitative formula for doing so.

As noted above, the medial axis skeletonization method described herein may identify vessel segments (such as coronary artery segments) between vessel branch points. The injected contrast bolus travel in a series of angiographic image frames can be used by the computer to determine the direction of blood flow in a given coronary artery segment. In a standard left main coronary artery injection, the first coronary artery to receive the contrast bolus is the left main coronary artery. Coronary artery morphology statistics may be generated by the computer. For example, a graphical user interface (also referred to herein as a “GUI”) may be displayed by the computer in which a separate screen on the graphical user interface may display a histogram of coronary artery diameter, either in the aggregate or subdivided by coronary artery segment. To determine if coronary artery stenosis, the initial 10% of length of a coronary artery may be taken as the baseline. If the subsequent 90% of the length on a non-left main coronary artery dips below a 70% reduction, then the computer may flag the segment in the graphical user interface (or some other rendering of the image) for special attention by the interpreting cardiologist. An example flagging is the yellow arrow pointing to the right at a suspected stenosis in FIG. 5H. The bias of the statistical system is preferably to have excess sensitivity, and to bring to human cardiology interpretation those areas that might need extra consideration for placement of a stent. The final decision on the necessity of a stent is preferably left to the interpreting cardiologist.

Information regarding coronary artery stents is typically published in catalogues by their respective manufacturers. Additionally, particular stents available at a health care facility may also be listed in the facility's inventory and/or billing systems. In an example embodiment, if the computer identifies a suspected stenosis, it may retrieve information about suitable stents from one or more of the foregoing sources, or from a database, or the user may enter information about available stents, and the computer may generate a GUI in which a selection of one or more stents is displayed alongside an image of the vessel.

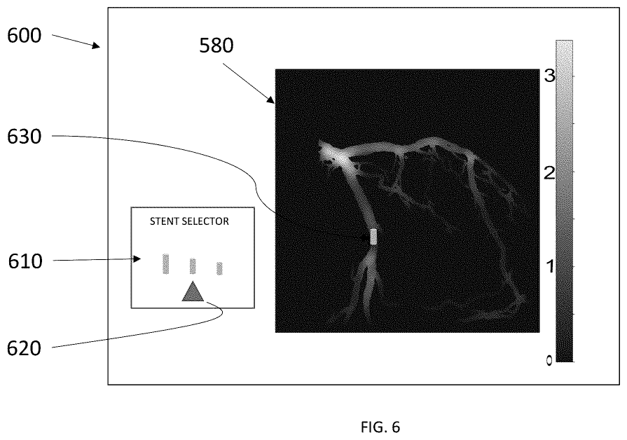

FIG. 6 illustrates an example of a GUI 600 that may be generated by the computer to present a selection of one or more stents 610 to a user (e.g., a cardiologist). In this example, the GUI displays a selection of stents 610 in combination with the heat map 580 described above. In an embodiment, the size of the stents 610 in GUI 600 may be displayed to the same scale as the vessels in the heat map 580 so that accurate size comparisons may be made by the user. In an embodiment, information about the stents 610 (e.g., brand and/or dimensions) may be displayed in the GUI. For example, stent information may be displayed in a pop-up window by clicking on or hovering over one of the stents using a suitable input device (such as a touchscreen, a mouse, or a trackball). In an embodiment, the GUI 600 may provide drag and drop stent selection and simulation functionality. For example, the user may select one of the stents 610 in the GUI using a suitable input device, drag the stent over the heat map 580, and drop the stent onto the stenotic artery segment for visual inspection. Since the stents and the vessels are displayed to the same scale, if the stent fits, then it may be selected for actual implantation by the user. For example, the GUI may display options to finalize or undo the selection when the user hovers over or clicks on the stent that was dragged onto the image. An example result of a drag and drop operation using the GUI is shown in FIG. 6 in green at 630. This adornment of the GUI with coronary stent options aids the judgment of the interpreting cardiologist, but the cardiologist is responsible for the decision. The GUI may identify the stent selected, for example as “Manufacturer Name 20 mm length by 3 mm”. In an embodiment, the computer may communicate this information with the hospital inventory and/or billing systems via an electronic communications network.

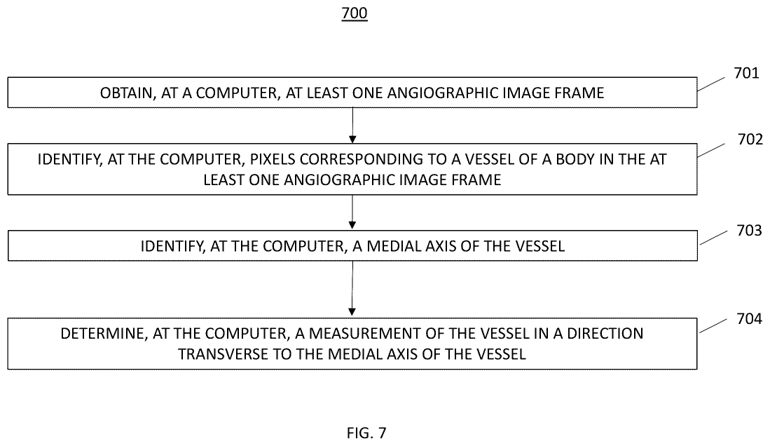

FIG. 7 is a flowchart 700 illustrating a method for measuring vessels in a body, according to an example embodiment. At operation 701, at least one angiographic image frame is obtained at a computer. For example, the angiographic image frame(s) may be obtained using the rotational x-ray system 28 (FIGS. 1A and 1B) and acquired or received by a computer in the rotational x-ray system 28 or by a standalone computer (e.g., image reconstructor 65 and/or computer 66).

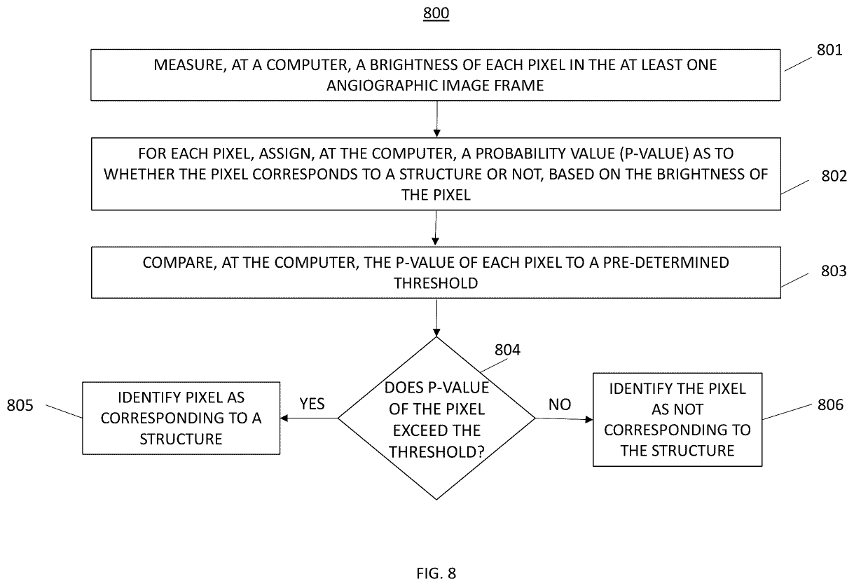

At operation 702, pixels corresponding to a vessel of the body are identified in the at least one angiographic image frame. An example method for performing this operation is shown in FIG. 8.

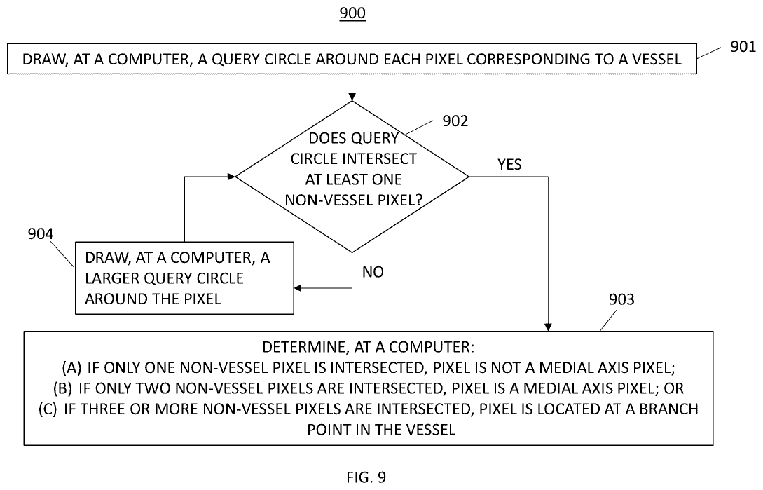

At operation 703, the computer identifies a medial axis the vessel. An example method for performing this operation is shown in FIG. 9.

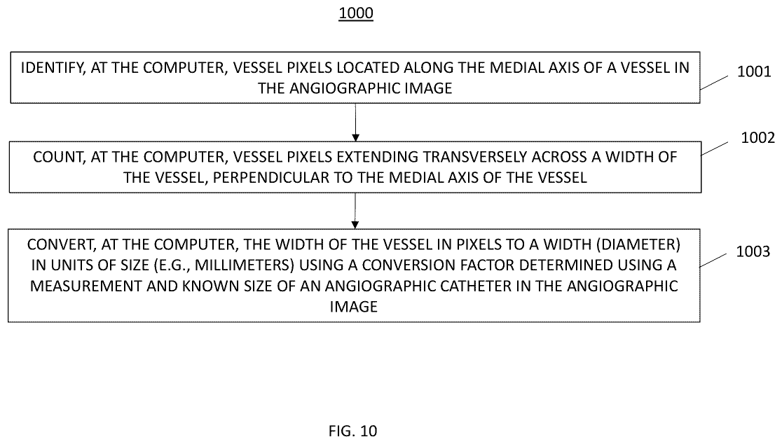

At operation 704, the computer determines a measurement of the vessel in the at least one angiographic image. An example method for performing this operation is shown in FIG. 10.

FIG. 8 is a flow chart 800 illustrating a method for identifying pixels that correspond to a vessel according to an example embodiment. At operation 801, the computer measures or otherwise determines a brightness or intensity of each pixel in the at least one angiographic image.

At operation 802, the computer assigns, for each pixel, a probability value (p-value) as to whether the pixel corresponds to a vessel or not. As described above, the p-value may be assigned based on the brightness or intensity of the pixel. In an example embodiment, this operation may be performed by a neural network trained to identify pixels corresponding to a vessel in an angiographic image.

At operation 803, the computer may compare each p-value against a predetermined threshold.

At operation 804, a determination is made whether or not the p-value of a pixel exceeds the threshold, based on the comparison. If the p-value exceeds the threshold, the computer may identify the pixel as corresponding to a vessel in operation at operation 805. Conversely, if the p-value of a pixel is less than or equal to the threshold, the computer may identify the pixel as a non-vessel at operation 806. The threshold may be static (e.g., a fixed number) or dynamic (e.g., determined based on one or more factors such as an average intensity of all or some pixels in the at least one angiographic image frame).

FIG. 9 is a flowchart 900 illustrating a method of identifying a medial axis of a vessel, according to an example embodiment.

At operation 901, for every pixel determined to be a vessel pixel, the computer draws a tiny circle (also referred to as a “query circle”) around the vessel pixel. In one example, the first query circle may be a group of pixels circumscribing the vessel pixel.

At operation 902, the computer determines whether or not the query circle intersects one or more non-vessel pixels. If the query circle does not intersect at least one non-vessel pixel, the size of the query circle may be increased at operation 904 and operation 902 may be performed again. This process may be repeated until the query circle intersects at least one non-vessel pixel, at which time the method proceeds to operation 903.

At operation 903, the computer determines one of the following: (a) if, at a certain diameter, the query circle intersects a single non-vessel pixel, the vessel pixel at the center of the query circle is determined to not be a medial axis pixel; (b) if at a certain diameter, the query circle intersects two non-vessel pixels, the vessel pixel at the center of the query circle is considered to be a medial axis pixel; or (c) if at a certain diameter, the query circle intersects three or more non-vessel pixels, the vessel pixel at the center of the query circle is considered to be located at a branch point of the vessel. In an example embodiment, the coordinates (e.g., row, column) of every medial axis pixel may be recorded. The list of coordinates defines the medial axis of the segmented vessel. The diameter in pixels of the corresponding query circle for each medial axis pixel (i.e., the diameter of the query circle that intersected two non-vessel pixels) may also be recorded.

FIG. 10 is a flowchart 1000 illustrating a method for measuring a diameter of a vessel in pixels, according to an example embodiment. At operation 1001, the computer may identify the vessel pixels located along the medial axis of a vessel in the at least one angiographic image. In an example embodiment, the computer may identify the medial axis pixels from a list of coordinates generated by the skeletonization process described above.

At operation 1002, the computer may determine the diameter of the vessel in pixels at one or more points along the medial axis of the vessel by counting the number of vessel pixels extending transversely across a width of the vessel, perpendicular to the medial axis of the vessel. In an example embodiment, this may be accomplished by superimposing the skeletonized version of the angiographic image over the segmented (binarized) version of the angiographic image, and counting the number of vessel pixels in a transverse direction relative to the medial axis of the vessel.

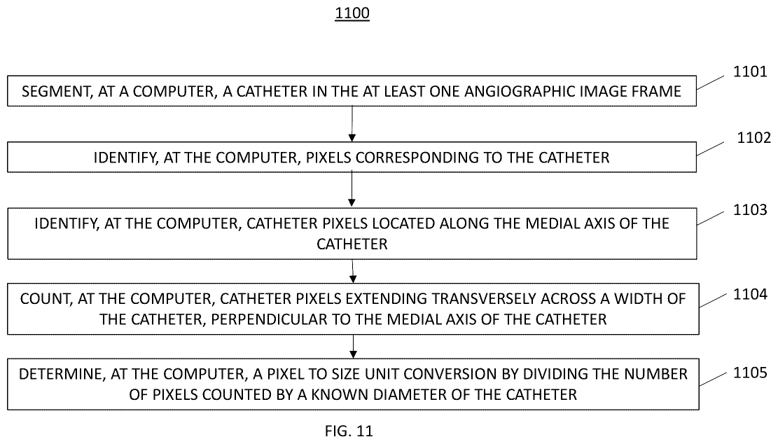

At operation 1003, the computer may convert the diameter of the vessel in pixels to a diameter in units of size or dimension (such as millimeters). In an example embodiment, this operation may be performed using the conversion factor determined by the method shown in FIG. 11.

FIG. 11 is a flowchart 1100 illustrating a method of determining a conversion factor for converting a diameter of a vessel in pixels to a diameter of the vessel in units of size or dimension (such as millimeters), according to an example embodiment.

At operation 1101, the computer may segment a catheter in the at least one angiographic image frame. For example, a neural network may be used to identify the pixels in the at least one angiographic image frame corresponding to the catheter (referred to herein as “catheter pixels”) at operation 1102.

At operation 1103, the computer may identify the catheter pixels that are located along the medial axis of the catheter, e.g., using the medial axis skeletonization technique described herein.

At operation 1104, the computer may count the number of catheter pixels extending transversely across a width of the catheter, perpendicular to the medial axis of the catheter.

At operation 1105, the computer may determine a pixel to size unit conversion factor by dividing the number of pixels counted by a known diameter of the catheter. The conversion factor may then be used to convert the diameter of a vessel in pixels to a diameter of the vessel is units of size or dimension (such as millimeters).

The present invention may include a method, system, device, and/or a computer program product at any possible technical detail level of integration. The computer program product may include a computer readable storage medium (or media) having computer readable program instructions thereon for causing a processor to carry out aspects of the present invention.

Computer readable program instructions described herein can be downloaded to respective computing/processing devices from a computer readable storage medium or to an external computer or external storage device via a network, for example, the Internet, a local area network, a wide area network and/or a wireless network. The network may comprise conductive transmission cables, optical transmission fibers, wireless transmission, routers, firewalls, switches, gateway computers and/or edge servers. A network adapter card or network interface in each computing/processing device may receive computer readable program instructions from the network and forward the computer readable program instructions for storage in a computer readable storage medium within the respective computing/processing device.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The above description is for the purpose of teaching the person of ordinary skill in the art how to practice the subject of the present application, and it is not intended to detail all those obvious modifications and variations of it which will become apparent to the skilled worker upon reading the description. For example, techniques other than the neural network example described herein may be used to identify pixels corresponding to a vessel in the at least one angiographic frame. In one example, instead of basing the identification on a comparison of a p-value of each pixel with a threshold p-value, the identification may be based on a comparison of another value associated with each pixel, such as brightness or intensity, with a threshold value. In another example, instead of identifying pixels with values exceeding a threshold as vessel pixels, the techniques may be modified to identify as vessel pixels those pixels with values equal to or exceeding a threshold value, equal to or less than a threshold value, or less than a threshold value. In yet another example, a wavefront analysis may be used to identify pixels that correspond to vessels in an angiographic image frame. In the wavefront analysis embodiment, at least one angiographic image frame is obtained, and the position and orientation of all edges in the angiographic image are determined using a form of wavefront analysis (also referred to as wavefront set analysis or micro local harmonic analysis). The wavefront analysis may utilize an underlying analysis called shearlet analysis which may involve fitting shearlets to find the edges of a vessel in the angiographic image. One edge may be identified where the wavefront transitions from positive to negative at all frequencies, and the other edge may be identified where the wavefront transitions from negative to positive at all frequencies.

In a further modification, instead of using the query circle method to measure a dimension transverse to the medial axis of a vessel, the computer may determine a slope of the medial axis at each medial axis pixel (e.g., using coordinates of the medial axis pixel and adjacent medial axis pixels), determine a direction that is transverse to the slope, and count the number of vessel pixels between non-vessel pixels in the direction that is transverse to the slope.

In yet another modification, instead of generating a graphical user interface (GUI) with vessel measurement and stenosis information, embodiments herein may generate an image data file compliant with the Digital Imaging and Communications in Medicine (DICOM) standard, which is currently the standard image data format in radiology. The metadata in the DICOM format includes an overlay field that can hold pixel data (e.g., a bitmap). In this example embodiment, a DICOM-compliant overlay file may be generated that a DICOM-compliant commercial GUI can render at a user's choice. For example, the overlay file may include a bitmap containing at least one of the following graphical elements: a scale for measuring vessel dimensions, stenosis measurements, arrows (e.g., pointing to a suspected stenosis), numbers, and stent size proposals. The overlay file may be configured to be overlayed on the angiographic image frame (like a mask) when displayed, so that the information in the overlay file may be viewed in conjunction with vessels in the angiographic image. Preferably, the overlay file is configured to match the scale of the angiographic image frame so that, for example, an arrow in the overlay file may point to a suspected stenosis in the angiographic image when a user chooses to show the overlay. To generate the vessel measurement and stenosis information as a DICOM-compliant overlay, the computer may save the overlay as a separate bitmap file or encode the bitmap directly into the overlay field of a DICOM file. For example, the computer may rasterize the information into the overlay DICOM field. DICOM-compliant software packages that a radiologist or a cardiologist might use typically have a checkbox on their GUIs that the radiologist or cardiologist can click to show the overlay. All hospitals and radiology units have a contract with a DICOM viewer company. It is generally part of the PACS (picture archiving and communication system) at the institution.

In their simplest form, the techniques described herein may be implemented as a cloud-based software-as-a-service (SaaS) that receives angiographic image frames from an originating entity over an electronic communications network (such as a local area network, a wide area network, and/or the Internet, etc.), performs vessel measurement, places the result in the overlay field of a DICOM-compliant file, and returns the DICOM-compliant file to the originating entity over the electronic communications network. An example data flow may comprise the following operations: (a) angiography suite performs an angiogram; (b) angiogram is received by computer as a DICOM format file (the DICOM format allows for including a family of images as are acquired in an angiogram. In the current standard, the images are in the PixelData field of the DICOM file); (c) computer records the address of the sending system; (d) computer performs a spatiotemporal transform of the angiographic data; (e) computer performs vessel measurements, identifies areas of stenosis, finds matching stents in stent catalogs; (f) computer creates a new DICOM format file in which vessel measurements, areas of stenosis, and related information are rendered into the Overlay field of the DICOM file (and, optionally, an enhanced version of the angiographic image frame—such as a segmented image, a spatiotemporally-transformed image, or an image sequence that is the substrate of the medial axis transform—is attached to the PixelData field of the same DICOM file; and (g) the resulting DICOM file is sent back to the originating sender. The computer may also keep a log of the entire transaction. The originating entity receives the DICOM file sent by the computer; and the radiologists and cardiologists may view the DICOM file on the computer screens using their DICOM-compliant viewers. When viewing the DICOM file, they may click on the Overlay button to show the vessel measurement data, etc., overlayed on the original angiographic image (or, alternatively, the spatiotemporally-transformed angiographic image) and unclick on the Overlay button to remove the overlayed items.

It is intended, however, that all such modifications and variations be included within the scope of the present invention, which is defined by the following claims. The claims are intended to cover the components and steps in any sequence which is effective to meet the objectives there intended, unless the context specifically indicates the contrary.

What is claimed is:

1. A method comprising:
obtaining, at a computer, at least one angiographic image frame;
identifying, at the computer, pixels corresponding to a vessel of a body in the at least one angiographic image frame;
determining, at the computer, a medial axis of the vessel; and
determining, at the computer, a diameter of the vessel in the at least one angiographic image frame by counting pixels corresponding to the vessel in a direction transverse to the medial axis of the vessel.

2. The method of claim 1, wherein determining a medial axis of the vessel comprises performing, at the computer, a medial axis skeletonization of the pixels corresponding to the vessel.

3. The method of claim 2, wherein:
performing the medial axis skeletonization comprises, for each pixel corresponding to a vessel, drawing, at the computer, a query circle around the pixel and determining, at the computer, whether the query circle intersects one or more pixels that do not correspond to a vessel.

4. The method of claim 3, wherein:
performing the medial axis skeletonization further comprises determining, at the computer, that a pixel is not a medial axis pixel if the query circle intersects only one pixel that does not correspond to a vessel.

5. The method of claim 3, wherein performing the medial axis skeletonization further comprises determining, at the computer, that a pixel is a medial axis pixel if the query circle intersects two pixels that do not correspond to a vessel.

6. The method of claim 3, wherein performing the medial axis skeletonization further comprises determining, at the computer, that a pixel is located a branch point of the vessel if the query circle intersects three or more pixels that do not correspond to a vessel.

7. The method of claim 5, wherein determining a measurement of the vessel further comprises generating a segmented image of the vessel using the pixels corresponding to the vessel; and
superimposing, at the computer, the medial axis of the vessel and the segmented image of the vessel.

8. The method of claim 7, wherein determining a measurement of the vessel further comprises recording, at the computer, a diameter the query circle that intersected two pixels not corresponding to a vessel as a diameter of the vessel.

9. The method of claim 1, further comprising converting, at the computer, the diameter of the vessel in pixels to a diameter in size units.

10. The method of claim 1, further comprising rendering, at the computer, a colored map of the vessel in which a color and hue of the vessel corresponds to a diameter of the vessel.

11. The method of claim 1, further comprising detecting, at the computer, a stenosis in the vessel.

12. The method of claim 11, further comprising generating, at the computer, a graphical user interface displaying at least one stent in combination with an image of the vessel.

13. The method of claim 12, wherein the at least one stent and the image of the vessel are displayed at the same scale.

14. The method of claim 12, wherein the graphical user interface permits the at least one stent to be dragged and dropped onto the image of the vessel.

15. The method of claim 1, further comprising generating an overlay file configured to be superimposed over and viewable together with the at least one angiographic image frame, the overlay file containing at least one graphical element selected from the group consisting of a dimensional scale for vessel measurement, an arrow, a number, and stent information.

16. The method of claim 15, wherein the at least one graphical element is configured to be displayed at the same scale as the at least one angiographic image frame.

17. A system comprising:
one or more computer processors;
one or more computer readable storage media;
program instructions stored on the one or more computer readable storage media for execution by at least one of the one or more computer processors, the program instructions comprising instructions to:
obtain at least one angiographic image frame;
identify pixels corresponding to a vessel of a body in the at least one angiographic image frame;
determine a medial axis of the vessel; and
determine a diameter of the vessel in the at least one angiographic image frame by counting pixels corresponding to the vessel in a direction transverse to the medial axis of the vessel.

18. The system of claim 17, wherein the instructions to determine a medial axis of the vessel comprise instructions to perform a medial axis skeletonization of the pixels corresponding to the vessel.

19. The system of claim 18, wherein the instructions to perform a medial axis skeletonization comprise instructions to draw, for each pixel corresponding to a vessel, a query circle around the pixel and determine whether the query circle intersects one or more pixels that do not correspond to a vessel.

20. The system of claim 19, wherein the instructions to perform the medial axis skeletonization further comprise instructions to determine, at the computer, that a pixel is not a medial axis pixel if the query circle intersects only one pixel that does not correspond to a vessel.

21. The system of claim 19, wherein the instructions to perform the medial axis skeletonization further comprise instructions to determine, at the computer, that a pixel is a medial axis pixel if the query circle intersects two pixels that do not correspond to a vessel.

22. The system of claim 19, wherein the instructions to perform the medial axis skeletonization further comprise instructions to determine, at the computer, that a pixel is located at a branch point of the vessel if the query circle intersects three or more pixels that do not correspond to a vessel.

23. The system of claim 21, wherein the instructions to determine a measurement of the vessel further comprise instructions to generate a segmented image of the vessel using the pixels corresponding to the vessel; and
superimpose, at the computer, the medial axis of the vessel and the segmented image of the vessel.

24. The system of claim 23, wherein the instructions to determine a measurement of the vessel further comprise instructions to record, at the computer, a diameter of the query circle that intersected two pixels not corresponding to a vessel as a diameter of the vessel.

25. The system of claim 17, wherein the instructions further comprise instructions to convert, at the computer, the diameter of the vessel in pixels to a diameter in size units.

26. The system of claim 17, wherein the instructions further comprise instructions to render, at the computer, a colored map of the vessel in which a color and hue of the vessel corresponds to a diameter of the vessel.

27. The system of claim 17, wherein the instructions further comprise instructions to detect, at the computer, a stenosis in the vessel.

28. The system of claim 27, wherein the instructions further comprise instructions to generate, at the computer, a graphical user interface displaying at least one stent in combination with an image of the vessel.

29. The system of claim 28, wherein the at least one stent and the image of the vessel are displayed at the same scale.

30. The system of claim 28, wherein the graphical user interface permits the at least one stent to be dragged and dropped onto the image of the vessel.

31. The system of claim 17, wherein the instructions further comprise instructions to generate an overlay file configured to be superimposed over and viewable together with the at least one angiographic image frame, the overlay file containing at least one graphical element selected from the group consisting of a dimensional scale for vessel measurement, an arrow, a number, and stent information.

32. The system of claim 31, wherein the at least one graphical element is configured to be displayed at the same scale as the at least one angiographic image frame.

33. One or more non-transitory computer readable storage media encoded with instructions that, when executed by a processor, cause the processor to:
obtain at least one angiographic image frame;
identify pixels corresponding to a vessel of a body in the at least one angiographic image frame;
determine a medial axis of the vessel; and
determine a diameter of the vessel in the at least one angiographic image frame by counting pixels corresponding to the vessel in a direction transverse to the medial axis of the vessel.

34. The computer readable storage media of claim 33, wherein the instructions to determine a medial axis of the vessel comprise instructions that, when executed by a processor, cause the processor to perform a medial axis skeletonization of the pixels corresponding to the vessel.

35. The computer readable storage media of claim 34, wherein the instructions to perform a medial axis skeletonization comprise instructions that, when executed by a processor, cause the processor to draw, for each pixel corresponding to a vessel, a query circle around the pixel and determine whether the query circle intersects one or more pixels that do not correspond to a vessel.

36. The computer readable storage media of claim 35, wherein the instructions to perform the medial axis skeletonization further comprise instructions that, when executed by a processor, cause the processor to determine, at the computer, that a pixel is not a medial axis pixel if the query circle intersects only one pixel that does not correspond to a vessel.

37. The computer readable storage media of claim 35, wherein the instructions to perform the medial axis skeletonization further comprise instructions that, when executed by a processor, cause the processor to determine, at the computer, that a pixel is a medial axis pixel if the query circle intersects two pixels that do not correspond to a vessel.

38. The computer readable storage media of claim 35, wherein the instructions to perform the medial axis skeletonization further comprise instructions that, when executed by a processor, cause the processor to determine, at the computer, that a pixel is located at a branch point of the vessel if the query circle intersects three or more pixels that do not correspond to a vessel.

39. The computer readable storage media of claim 38, wherein the instructions to determine a measurement of the vessel further comprise instructions that, when executed by a processor, cause the processor to generate a segmented image of the vessel using the pixels corresponding to the vessel; and
superimpose, at the computer, the medial axis of the vessel and the segmented image of the vessel.

40. The computer readable storage media of claim 39, wherein the instructions to determine a measurement of the vessel further comprise instructions that, when executed by a processor, cause the processor to record, at the computer, a diameter of the query circle that intersected two pixels not corresponding to a vessel as a diameter of the vessel.

41. The computer readable storage media of claim 33, wherein the instructions further comprise instructions that, when executed by a processor, cause the processor to convert, at the computer, the diameter of the vessel in pixels to a diameter in size units.

42. The computer readable storage media of claim 33, wherein the instructions further comprise instructions that, when executed by a processor, cause the processor to render, at the computer, a colored map of the vessel in which a color and hue of the vessel corresponds to a diameter of the vessel.

43. The computer readable storage media of claim 33, wherein the instructions further comprise instructions that, when executed by a processor, cause the processor to detect, at the computer, a stenosis in the vessel.

44. The computer readable storage media of claim 43, wherein the instructions further comprise instructions that, when executed by a processor, cause the processor to generate, at the computer, a graphical user interface displaying at least one stent in combination with an image of the vessel.

45. The computer readable storage media of claim 44, wherein the at least one stent and the image of the vessel are displayed at the same scale.

46. The computer readable storage media of claim 44, wherein the graphical user interface permits the at least one stent to be dragged and dropped onto the image of the vessel.

47. The computer readable storage media of claim 33, wherein the instructions further comprise instructions that, when executed by a processor, cause the processor to generate an overlay file configured to be superimposed over and viewable together with the at least one angiographic image frame, the overlay file containing at least one graphical element selected from the group consisting of a dimensional scale for vessel measurement, an arrow, a number, and stent information.

48. The computer readable storage media of claim 47, wherein the at least one graphical element is configured to be displayed at the same scale as the at least one angiographic image frame.
